(19) United States
(12) Patent Application Publication
(10) Pub. No.: US 2021/0038357 A1
(43) Pub. Date: Feb. 11, 2021

(54) ORAL CARE ILLUMINATION DEVICE WITH BACKLIGHT MODULE AND METHOD OF USE

(71) Applicant: KONINKLIJKE PHILIPS N.V., EINDHOVEN (NL)

(72) Inventor: TIANYI WANG, KENMORE, WA (US)

(21) Appl. No.: 16/963,962

(22) PCT Filed: Jan. 21, 2019
(86) PCT No.: PCT/EP2019/051321

§ 371 Date: Jul. 22, 2020

Related U.S. Application Data
(60) Provisional application No. 62/620,117, filed on Jan. 22, 2018.

(51) Int. Cl. A61C 19/06; A61N 5/06

(52) U.S. Cl. CPC A61C 19/066; A61N 2005/0606; A61N 5/0601; A61N 5/062

(57) ABSTRACT

An oral care illumination device (30) includes a mouthpiece (300) and a backlight module (320). The mouthpiece is adapted to fit at least a portion of a user's mouth. The backlight module is coupled to the mouthpiece and includes at least one light source (320), a light guide plate (310) configured to receive light from the at least one light source, including a curved light emitting surface (360) and a plurality of microstructures (350), and at least one reflector (340) to redirect light through the light guide plate. The plurality of microstructures, the curved light emitting surface, and the at least one reflector alter a propagation of light from the at least one light source through the light guide plate to illuminate a predetermined area of the user's mouth with a desired light distribution.

### CROSS REFERENCE TO RELATED APPLICATION

This application claims priority to U.S. Provisional Patent Application Ser. No. 62/620,117 filed 22 Jan. 2018, which is incorporated herein by reference in its entirety.

### FIELD OF THE INVENTION

The inventive subject matter relates to oral care illumination devices and backlight modules, such as used for teeth whitening or oral care, and further to methods of using such devices and modules.

### BACKGROUND

Some oral care devices use light for illuminating teeth. The light can be applied to the teeth and/or gums for different purposes. The teeth and/or gums may be illuminated for the purpose of whitening teeth, removal of plaque or bacteria, or other oral hygiene or care uses. For example, teeth whitening can be accomplished by applying a whitening gel, varnish, or other oral care product onto the teeth and activating the product by light of a specific wavelength. For the treatment to be effective, the light intensity needs to be sufficiently high. It is also desirable that the light intensity is uniformly distributed over the treatment area. In order to avoid damage to the pulp in the teeth it is also important to keep teeth heating safely below a critical temperature.

Conventional illumination techniques and devices are often costly and/or require significant space to implement. Such devices are also limited by the type and/or number of light sources used. As a result, the existing solutions for achieving uniform light distribution usually preclude their use in oral healthcare products.

Accordingly, there is a need for oral care illumination devices and methods that can illuminate a specified area in a user's mouth with a sufficiently high light intensity, with a uniform light distribution, and which, alternatively or additionally, do not cause unacceptable or dangerous heating of the teeth.

### SUMMARY OF INVENTION

The disclosed subject matter solves these and other problems by providing oral care illumination devices, as well as backlight modules for illumination of teeth in a user's mouth, and related methods.

In one representative embodiment, an oral care illumination device includes a mouthpiece adapted to fit at least a portion of a user's mouth, a backlight module coupled to the mouthpiece including at least one light source, a light guide plate configured to receive light from the at least one light source, and at least one reflector to redirect light through the light guide plate. The light guide plate includes a curved light emitting surface and a plurality of microstructures. The plurality of microstructures, the curved light emitting surface, and the at least one reflector alter a propagation of light from the at least one light source through the light guide plate to illuminate a predetermined area of the user's mouth with a desired light distribution.

In some embodiments, the mouthpiece includes an optically transparent teeth part allowing propagation of light from the light emitting surface of the light guide plate to the predetermined area of the user's mouth. In some embodiments, the plurality of microstructures are configured to project a uniform distribution of light on the predetermined area of the user's mouth. In other embodiments, the plurality of microstructures are configured to project a customized distribution of light on the predetermined area of the user's mouth. In some embodiments, the light source is housed within the mouthpiece. In other embodiments, the light source is mounted on a lateral side of the light guide plate. In further embodiments, the light source is located on a front side of the backlight module. In some embodiments, the plurality of microstructures are uniformly distributed over at least one light receiving surface of the light guide plate. In other embodiments, the plurality of microstructures are variably distributed over at least one light receiving surface of the light guide plate. In some embodiments, the plurality of microstructures includes microstructures that are hemisphere shaped. In some embodiments, the oral care illumination device further includes a brightness enhancement film disposed adjacent the curved light emitting surface of the light guide plate to improve light angular uniformity.

In another representative embodiment, a backlight module for homogenous illumination of teeth in a user's mouth is provided. The backlight module includes at least one light source arranged to deliver light of a predetermined wavelength and a light guide plate including a light emitting surface having a curvature complementary to at least a dental arch of the user's mouth. The light guide plate further includes a plurality of microstructures on a light receiving surface of the light guide plate redirecting light received from the at least one light source to the light emitting surface. At least one reflector can be disposed adjacent to the light guide plate to redirect light emitted by the at least one light source through the light guide plate to the light emitting surface. The plurality of microstructures, the curved light emitting surface, and the at least one reflector alter a propagation of light from the at least one light source and illuminate the dental arch of the user's mouth with light having a desired light distribution.

In some embodiments, the at least one light source includes an edge-lit backlight light source. In other embodiments, the at least one light source includes a direct backlight light source. In some embodiments, the at least one light source is a light emitting diode. In further embodiments, the backlight module includes a brightness enhancement film disposed adjacent a light emitting surface of the light guide plate to improve light angular uniformity. In some embodiments, the plurality of microstructures are uniformly distributed over at least one light receiving surface of the light guide plate. In other embodiments, the plurality of microstructures are variably distributed over at least one light receiving surface of the light guide plate. In some embodiments, the plurality of microstructures includes microstructures that are hemisphere shaped.

The inventive subject matter is also directed to a method of illuminating teeth, including providing an oral care illumination device as described above, positioning the oral care illumination device in the mouth of the user, activating the light source of the oral care illumination device and illuminating the teeth for a predetermined time.

One advantage of the inventive subject matter described herein is that a backlight module with a curved light guide plate or other light directing element can solve the problem of non-uniformity with light sources within tight space constraints. For example, a mouthpiece with a backlight module can be made very thin compared to conventional assembly solutions for teeth illumination. Another advantage is that the disclosed backlight modules can be manufactured of low-cost materials and components compared to existing light guide solutions for teeth whitening. Yet another advantage of the inventive subject matter described herein is that the curved backlight module provides the flexibility to modify the final output for different surface curvatures of the teeth and mouth, enabling light performance that is customized to the user in each product manufactured.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the inventive subject matter.

FIG. 1 is a perspective view of an exemplary embodiment of an oral care illumination device on a dental model.

FIG. 2A is a perspective view of another exemplary embodiment of an oral care illumination device.

FIG. 2B is a cut-away side view of an arrangement of an oral care illumination device such as shown in FIG. 2A when it is placed in the mouth of a user.

FIG. 3 is a perspective view of an another exemplary arrangement an oral illumination device.

FIG. 4 is an exploded view of an exemplary embodiment of a curved backlight module with an edge-lit configuration.

FIG. 5 is an exploded view of another exemplary embodiment of a curved backlight module with a direct backlight configuration.

FIG. 6 is a schematic representation illustrating portions of the curved backlight module including a uniform distribution of hemisphere shaped microstructures.

FIG. 7 is an exploded planar view of layers of the backlight module of FIG. 4.

FIG. 8 is a schematic representation illustrating portions of an embodiment of a backlight module including a variable distribution of microstructures on a light receiving surface.

FIG. 9 is a top view of the palate and lower dental arch fitted with an example embodiment of a backlight module and illustrates light distribution on the teeth.

### DETAILED DESCRIPTION OF EMBODIMENTS

The disclosed subject matter will become better understood through review of the following detailed description in conjunction with the figures. The detailed description and figures provide example embodiments of the invention described herein. Those skilled in the art will understand that the disclosed examples may be varied, modified, and altered without departing from the scope of the invention described herein.

Throughout the following detailed description, various examples of oral care devices, backlight modules and related oral care methods are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature or example.

The present disclosure describes various embodiments of devices and methods to provide homogenous illumination to a specific area in a mouth. By producing homogenous light from an adjustable number of light sources and using a curved backlight module with a light guide plate, or other light directing element, the disclosed subject matter provides a low-cost and efficacious lighting system for oral care treatments. In some embodiments, such as implementations used for teeth whitening procedures, homogenous illumination can be used to activate a reactive bleaching agent on the teeth. Oral care illumination devices described herein can be consumer products for home use or commercial products used by a professional; in both cases the user, i.e., a home user or a patient, is the user that receives the illumination from the device.

In some embodiments, an oral care illumination device can comprise a mouthpiece and a backlight module. The mouthpiece can be adapted to fit at least a portion of a user's mouth. The backlight module can be coupled to the mouthpiece and include a light source, a light guide plate, and a reflector. The light guide plate is configured to receive light from the light source and includes a curved light emitting surface and a plurality of microstructures. The microstructures and the reflector redirect light received from the light source to the curved light emitting surface. The combination of the microstructures, the curvature of the light emitting surface, and the reflector alters the propagation of light from the light source traveling through the light guide plate and emitted by the light emitting surface resulting in homogenous illumination of a predetermined area of the user's mouth with a desired light distribution.

The term “light source” should be understood to refer to any one or more of a variety of electromagnetic radiation sources, including, but not limited to, light-emitting diode (LED) based sources (including one or more LEDs as defined herein), incandescent sources (e.g., filament lamps, halogen lamps), fluorescent sources, phosphorescent sources, high-intensity discharge sources (e.g., sodium vapor, mercury vapor, and metal halide lamps), lasers, and other types of electroluminescent sources.

The term “LED” (light emitting diode) should be understood to include any electroluminescent diode, or other type of carrier injection or junction-based system that is capable of generating radiation in response to an electric signal. Thus, the term LED includes, but is not limited to, various semiconductor-based structures that emit light in response to current, light emitting polymers, organic light emitting diodes (OLEDs), electroluminescent strips, and the like. It should also be understood that the term LED does not limit the physical and/or electrical package type of an LED.

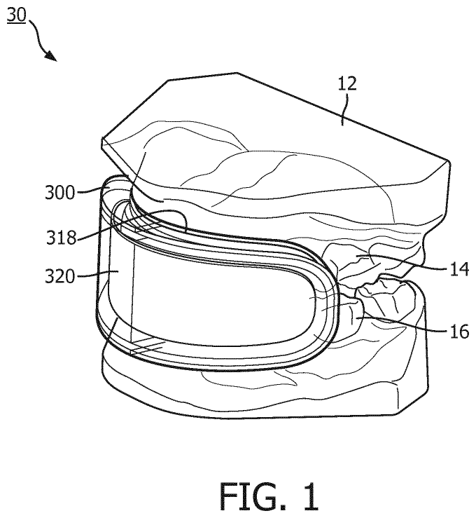

FIG. 1 shows an oral care illumination device 30 applied on a dental model 12 simulating the position of the device in the mouth of a user. The oral care illumination device 30 has a mouthpiece 300 and a backlight module 320. The mouthpiece 300 is placed in an area corresponding to the labial vestibule of the mouth of a user, thereby keeping lip tissue away from the tooth and gum surfaces. The mouthpiece allows positioning of the backlight module to provide optimal exposure of a portion of the user's mouth. As shown, the upper and lower frontal teeth and gum tissues that surround the teeth and covers the alveolar parts of the jaws can be illuminated.

Mouthpiece 300 has a curved portion 318 that is bent along an outer surface of the teeth on the upper and lower dental arches of dental model 12. Specifically, curved portion 318 of mouthpiece 300 fits both an upper dental arch 14 and lower dental arch 16 of dental model 12. In this position, the mouthpiece 300 can project light received from the backlight module 320 onto both upper and lower dental arches covering at least an area including the upper and lower incisors, cuspids, and first bicuspids.

Mouthpiece 300 can be provided with several light sources, for example suitable for activating a reactive agent. Some embodiments include light sources providing illumination from within the mouthpiece. Other embodiments may include light sources placed outside the mouthpiece and operably coupled to the mouthpiece such that the light source projects light onto a curved backlight module inside the mouthpiece, for example through a waveguide as can be provided by one or more optical fibers. By delivering light from an outside light source to the backlight module thermal management in the mouthpiece can be minimized. In further embodiments, oral care illumination devices may have direct backlight or edge-lit backlight modules, as described below, or both, or some other form of light source.

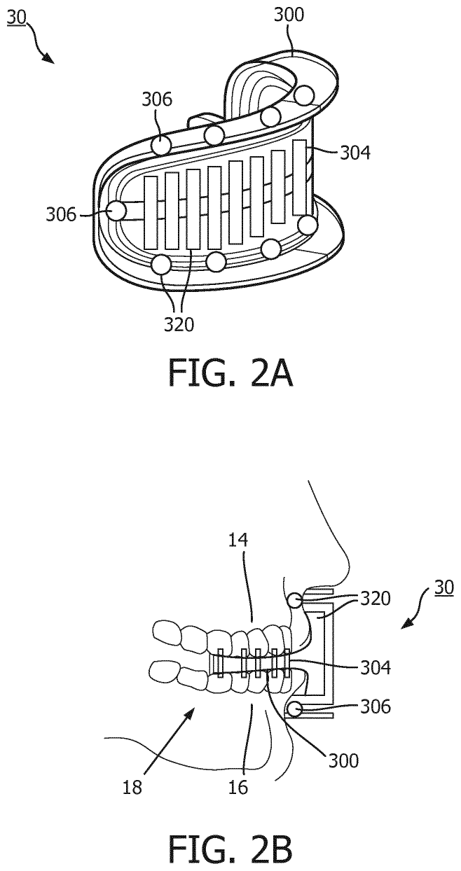

FIGS. 2A and 2B illustrate an example embodiment wherein an oral care illumination device 30 has a backlight module 320 integrated with a mouthpiece 300. Oral care illumination device 30 incorporates two variations of light sources, namely direct backlight illumination and edge-lit illumination. Direct backlight illumination is illustrated by direct light sources 304, illustrated as vertical lines in FIG. 2A. Edge-lit illumination is accomplished via side light sources 306, illustrated by dots in FIG. 2A. Some of the light emitted by light sources 304 and 306 travels through backlight module 320 to illuminate a predetermined area of the mouth. For example, as shown in FIG. 2B, oral care illumination device 30 can project light onto the teeth and gums of the upper dental arch 14 and lower dental arch 16 of a user's mouth 18 via backlight module 320. In other embodiments, the oral care illumination device can illuminate selective portions of the mouth, which can be on the facial side of the dental arches and/or on the lingual side of the dental arches, as well as on the dental arches of both the upper and lower jaw, or on only one of these arches, or on another portion of the mouth of a user.

Depending on the application, the desired light distribution, type and number of light sources can be adjusted. For example, tooth whitening applications may require light to be emitted at a predetermined wavelength to activate bleaching agents on the tooth surfaces. Typically, higher power density corresponds to faster reaction time of the bleaching agent, however the power density should not exceed safety limits for pulpal tissue. For some embodiments described herein, the power density (irradiance) of light on the teeth is uniform in the range of 0-200 mW/cm², preferably in the range of 25-50 mW/cm². Some embodiments may use light with a wavelength of 440-460 nm. Other embodiments can use for example blue light having a wavelength of 400-495 nm.

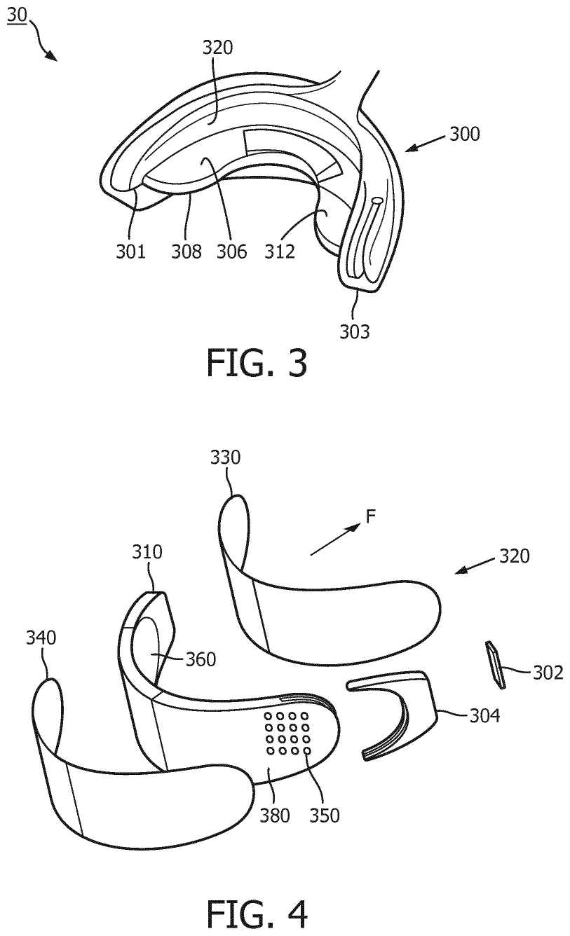

FIG. 3 shows another example embodiment wherein an oral care illumination device 30 includes a mouthpiece 300 with an optically transparent teeth part 301 arranged to face an outer surface of the teeth when in use. Mouthpiece 300 is sized and shaped for comfortable insertion into the user's mouth and can have a handle for user convenience. Mouthpiece 300 houses and supports a backlight module 320. Backlight module 320 is dimensioned to fit within mouthpiece 300 and is held in place by mouthpiece 300.

In some embodiments, backlight module 320 can be permanently attached to and integrated with mouthpiece 300. In other embodiments, backlight module 320 can be detachably coupled to mouthpiece 300 and include, for example, replaceable or interchangeable components. The dimensions of the mouthpiece and/or backlight module can be adjusted depending on the type of user, for example differently sized mouthpieces can be developed for adults and children.

Light sources 320 can be arranged at various locations. For example, at the edge of the backlight module in an edge-lit configuration, and/or along a front surface plane of the backlight module in a direct backlight configuration. In further embodiments, light sources can be provided outside the backlight module whereby light can be delivered through a wave guide, such as an optical fiber. Light leaving backlight module 320 is transmitted from mouthpiece 300 to the teeth via the optically transparent teeth part 301 and can illuminate a facial side of the teeth and/or gum on both the upper and lower dental arch of the user. Optically transparent teeth part 301 does not have to be completely transparent but can be partially transparent or even translucent or partially translucent, so long as an acceptable amount of light is transmitted onto the teeth and/or gum surface and the acceptable amount of light depends on the type of oral procedure that is applied to the user.

Optionally, mouthpiece 300 may include a bite part 312 protruding from a tooth facing surface of mouthpiece 300. As shown in FIG. 3, bite part 312 extends from a concave surface of mouthpiece 300 and follows the inner curvature of mouthpiece 300 so that the user can bite onto bite part 312. An upper bite surface 306 of bite part 312 accommodates the upper occlusal side of the teeth and a lower bite surface 308 accommodates the lower occlusal side of the teeth thereby allowing the user to bite into bite surfaces 306, 308 and helping the mouthpiece 300 stay in place in the mouth. In some embodiments, bite part 312 can be coupled to or integrated with optically transparent teeth part 301.

Mouthpiece 300 may also have an optional rim 303. Rim 303 may be shaped to contact the gums of the user along upper and lower dental arches. A function of rim 303 can be to serve as a sealing structure for preventing teeth whitening gel or other dental substances from leaking out and to assist in retaining the dental substance at a location on the teeth.

FIG. 4 illustrates components and their arrangement of a backlight module 320 having edge-lit illumination. Backlight module 320 can be used as a stand-alone element or can be associated with a mouthpiece, for example with mouthpiece 300 described above. Backlight module 320 has an overall curved shape which fits the mouth of a user and consists of an arrangement of layers including a light guide plate 310, shown as a middle layer, a reflector 340 shown as a bottom layer, and a brightness enhancement film 330, shown as a top layer in FIG. 4. The layers each have concave shapes complementary to each other and can be stacked such that a light emitting surface 360 on light guide plate 320 and brightness enhancement film 330 face the teeth. Juxtaposed to the light emitting surface 360 is a light receiving surface 380 which faces the reflector 340. Reflector 340 redirects light that does not project directly towards the teeth to a light receiving surface 380 on the light guide plate 310. Light exiting a light source 302 has a divergent angle, a small portion of the light within the divergent angle can reach teeth directly, however, most of the light from the divergent angle is redirected by the light guide plate and then projects onto the teeth. A general direction of light projecting onto the teeth is indicated by the arrow F in FIG. 4.

Light emitting surface 360 is in contact with brightness enhancement film 330 to enhance light leaving the light emitting surface and to help ensure the light reaches angular uniformity, i.e., the angle at which the beam of emitted light reaches at the surface of the teeth, because the brightness enhancement film improves the on-axis luminance, in addition to spatial uniformity of the brightness on the surface of the teeth. Optionally, the backlight module may have an additional layer, such as a diffusion film to adjust the final optical performance.

The layers of backlight module 320 are each curved to complement each other and fit over the dental arches in the mouth of a user. In some embodiments, the light guide plate can be made of a hard material that may need to be curved, whereas the reflector and the brightness enhancement film can be made of a soft pliable material that can be bent in a suitable shape. The mouthpiece and backlight module are dimensioned to fit a predetermined area of the mouth of a user and can be made very thin. In some embodiments, the thickness of light sources, such as LEDs, can be less than 1 mm, and therefore the thickness of the light guide plate can be less than 1 mm. The individual layers can be formed to have corresponding dimensions such that layers can be closely stacked in a mouthpiece. As shown in FIG. 4, each layer has an elongated shape corresponding to the anatomical curvature of a mouth. In further embodiments, the backlight module can be curved to be complementary to an inner curvature of the dental arch, selective sections of the dental arch, or other portions of the user's mouth. The layers of backlight module 320 can fit into a recess, cavity or chamber of mouthpiece 300 designed to hold the backlight module. The layers of backlight module 320 may be coupled together by any suitable means before inserting into the mouthpiece. In other embodiments, the individual layers can be held together by suitable means and used as a stand-alone element for homogenous illumination of the teeth.

Backlight module 320 has a light source 302, such as an LED, mounted on a lateral side of light guide plate 310 to deliver light of a predetermined wave length. In other embodiments, light source 302 can be a laser. Light source 302 is mounted in housing 304 coupled to the light guide plate 310 such that the light emitted by light source 302 projects onto light receiving surface 380 of the light guide plate 310 and onto the reflector 340 when light source 302 is activated. Light source 302 can be coupled to the housing via any suitable means. For example, housing 304 may have a recess adapted to hold light source 302 such that light emitted by light source 302 is directed to light guide plate 310. As shown in FIG. 4, housing 304 is arranged along the lateral side of light guide plate 310 as a distinct part that is connected to light guide plate 310 via a suitable coupling mechanism while allowing light emitted by light source 302 to reach light guide plate 310 uninhibited. In other embodiments, the light source can be mounted at many different locations where the same functionality can be provided. Light source 302 can be operated via power sources such as batteries or other appropriate power supplies.

Light guide plate 310 distributes light received from light source 302 to a concave light emitting surface 360 by propagating light from light source 302 via internal reflection and refraction based on the design and location of microstructures 350. By doing so, the backlight module 320 transforms a light beam from light source 302 and diffuses the light over light emitting surface 360 to provide uniform light distribution to the teeth.

In some embodiments, the light guide plate may be made from polycarbonate and the optically transparent teeth element may be made from silicone. Alternative materials for the light guide plate 310 include polymethylmethacrylate (PMMA), cyclic-olefin polymers, cyclic-olefin copolymers, polyetherimide, styrene and polyesters like OKP-4, and the like.

To change an angle of incoming light rays from light source 302, light receiving surface 380 of light guide plate 310 is modified to include a series of microstructures 350. For example, an array of hemisphere shaped microstructures 350 in close proximity to each other can act as a plurality of tiny, convex lenses that facilitate the reflection and refraction of incoming light rays of the light source 302. As shown in FIG. 4, light guide plate 310 incorporates laser-induced microstructures 350 on light receiving surface 380. Dimensions of individual microstructures, distribution of the microstructures over the light receiving surface, and spacing between individual microstructures can be adjusted and tailored to provide homogenous illumination. Further details of the microstructures 350 are discussed below.

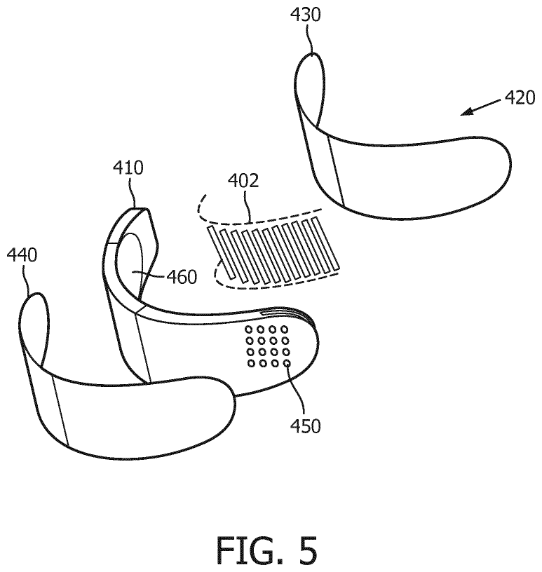

In another embodiment, illustrated in FIG. 5, a backlight module 420 includes a direct backlight illumination system. Backlight module 420 can be integrated with a mouthpiece, for example mouthpiece 300 described above, or used as a stand-alone or interchangeable element. Direct backlight module 420 includes light sources 402, shown as middle layer, and one or more light directing elements, such as a reflector 440, shown as a bottom layer, and a brightness enhancement film 430, shown as a top layer. Backlight module 420 has an overall shape that follows the curvature of the dental arches in the mouth, in a manner similar to the above-described embodiments. Light sources 402 can be installed or affixed on a front side of a light guide plate 410 facing the teeth when the mouthpiece is inserted in the mouth. Since light sources 402 are located at the front side, most of the light exiting light sources 402 can reach the teeth directly, however, light reflected from the teeth or light that did not reach the teeth can be redirected by reflector 440. Brightness enhancement film 430 may be used to help ensure the light reaches angular uniformity in addition to spatial uniformity at the surface of the teeth. In some embodiments, the light guide plate and/or brightness enhancement film can be omitted from the backlight module and similar functionality can be accomplished by other light directing elements.

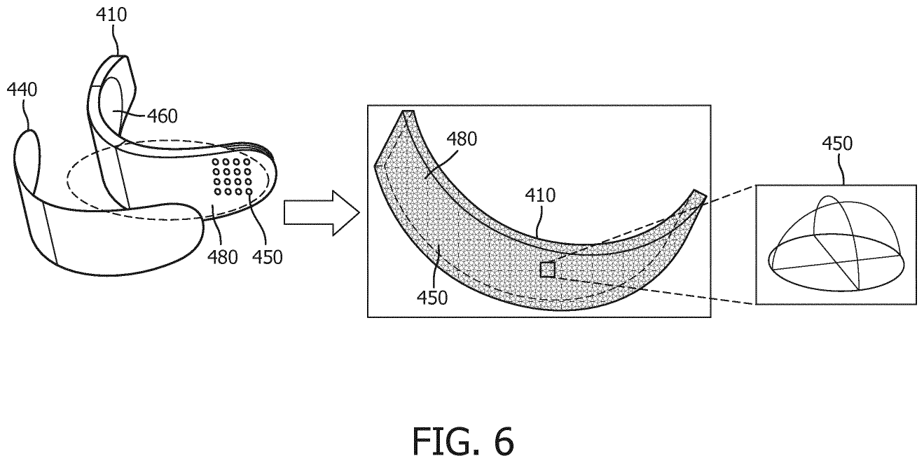

FIG. 6 shows a progressive enlargement of microstructures 450 on a light receiving surface 480 of light guide plate 410 facing reflector 440. Light receiving surface 480 of light guide plate 410 contains a uniform distribution of microstructures 450 wherein microstructures 450 are dispersed in a regular repeating pattern on light receiving surface 480. As further can be seen in FIG. 6, each single element of a microstructure 450 is formed as a hemisphere or dome shaped protrusion from the light guide plate 410. Other suitable shapes for microstructures include pyramid, sphere, or cube-shaped elements. Dimensions and shapes of individual microstructures 450 and spacing between microstructures 450 on light guide plate 410 can be adjusted and optimized. Some of the factors that may influence the selection of microstructure properties include the light source(s) utilized, the curvature of the light guide plate, the intended purpose of the oral care illumination device and a desired light distribution on a predetermined area of a user's mouth.

The term “microstructure” refers to any transformation created at a surface, or within the space, of a light directing element, such as a light guide plate, including any two dimensional and/or three dimensional transformation. Microstructures can be created by a variety of processes, some involving applying material to the surface, some involving removing material from the surface, or others involving a thermal-chemical surface reaction mechanism, for example, by melting. In other words, the microstructure size, spacing and/or patterns can be tailored according to the light source and desired light intensity at specific locations, and to compensate for curvature of the mouthpiece. The microstructures described herein can be implemented with any suitable laser marking or other technology such as injection molding, extruding, and/or embossing. For example, microstructures 450 can be created by laser-modification of a light receiving surface of light guide plate 410 or other light directing element provided to the backlight module. Some microstructures can be created by an interaction involving laser energy and the light directing element or some coating or finish applied to the surface of the light directing element. Alternatively, the texture of the surfaces of the light directing element can be modified by laser ablation or any other suitable process. Furthermore, one or more lasers can be used with a clear light directing element to add laser-induced modifications to the internal space of the light directing element.

The pattern of the microstructures 450 on the light guide plate 410 adjusts the angle of incoming light rays from the one or more light sources. Due to the modified configuration of the surface, optical reflection can occur at the surface of the light guide plate to redirect light rays. In addition to the optical reflection that can occur at the surface, the light can be further distributed, or refracted, as the light bounces off the reflector 440, and projects through the brightness enhancement film 430, such as shown in FIG. 5.

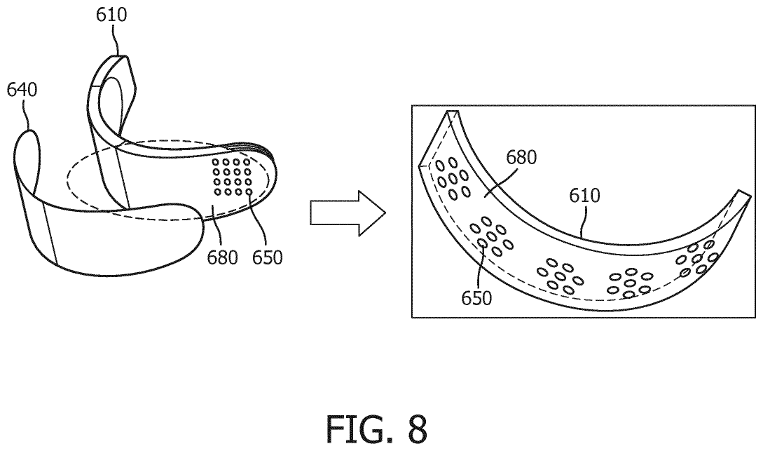

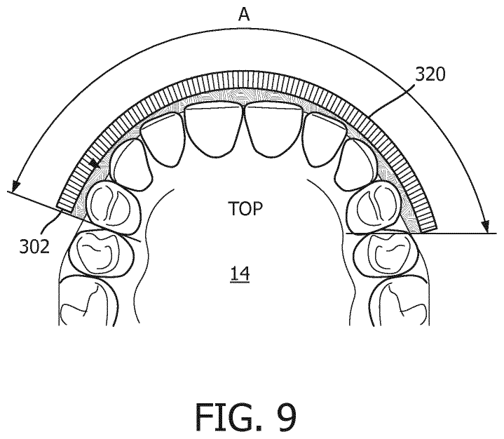

The microstructures can be applied to the light receiving surface in a uniform pattern, such as shown in FIG. 6. In other embodiments, shape and spacing of the microstructures on the light receiving surface of the light guide plate can be adjusted so that the backlight module projects a variable light intensity on a predetermined area of a user's mouth. For example, the distribution of light can be customized depending on the procedure for which the device is used and the individual requirements of a user, such as dimensions and curvature of the mouth. Microstructures can be applied in a non-uniform or variable patterns, depending on the number of light sources used, the desired light distribution, how light is to be reflected, and any other optical specifications and conditions to project a customized pattern of light intensity. In another embodiment, illustrated in FIG. 8, a light guide plate 610 is provided with microstructures 650 grouped along the light emitting surface 680 facing reflector 640 in a specific pattern to accomplish customized illumination of the teeth. Yet other embodiments, may include a variable or random distribution pattern of microstructures over light emitting surface 680.

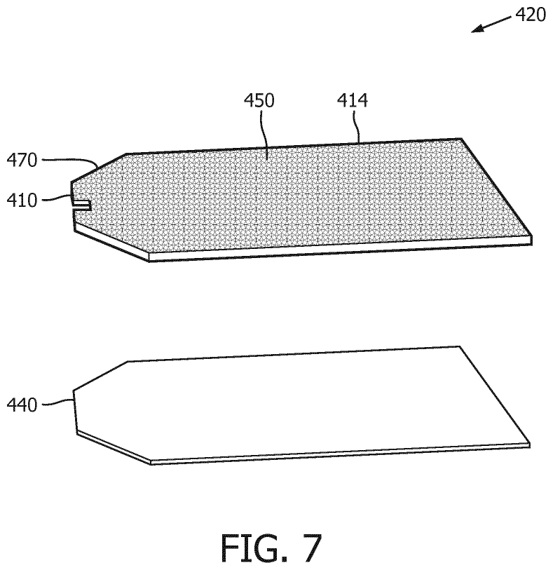

FIG. 7 shows an exploded view of the different layers of backlight module 420 in a planar surface. Backlight module 420 includes at least one reflector 440 that reflects light back toward the surface of the teeth. Microstructures 450 are incorporated into a light guide plate 410. In other embodiments, microstructures may be attached to one or more surfaces of the light guide plate. In further embodiments, the backlight module may have multiple reflectors, placed at varying distances from the light guide plate to optimize reflection of the light. Backlight module 420 may also include a diffusion film 470 on the light emitting surface of light guide plate 410 to help further increase the light angular uniformity. Light guide plate 410 may have a material or coating 414 around the outer edges thereof to contain and reflect light within the mouthpiece. The light guide plate with microstructures, the reflector, and the diffusion film, serve to mix the light rays to achieve an improvement of spatial and angular uniformity of light projected onto the surface of the teeth.

FIG. 9 is a top view of a dental model and an upper dental arch 14 fitted with a backlight module 320 and illustrates a light distribution pattern on the teeth as obtained according to an optical modelling procedure. The light distribution pattern shows how light rays emitted by light source 302 are directed to a predetermined area of the user's mouth, in particular indicated as area A in FIG. 9 and covering at least a portion of upper dental arch 14 including the incisors, cuspids, and first bicuspids. Optical models of light distribution provided by a specific design for a backlight module can be optimized according to an individual's location and shape of teeth or other structural specifications.

Illumination optimization for various teeth geometries can be verified by using optical design software to use sequential/non-sequential ray tracing to determine light distribution. Optical modeling of the backlight module, associated illumination, various microstructure sizes, patterns and spacing can be used to optimize the desired light output and location. By adjusting the size and spacing of the microstructures, and the pattern thereof, light distribution at the surface of the teeth and/or gums can be homogenized. After an optical model of the backlight module is optimized according to teeth geometry, design parameters can be input into a laser engraving machine for automated manufacturing, allowing large scale production, as well as customized design, at minimal time and cost.

By use of a light guide plate to optimize light irradiance distribution, more homogenous light distribution on a teeth surface can be achieved by means of optimization and customization of the dimensions of the microstructures and gap distance between microstructures.

The inventive subject matter further contemplates a method of illuminating teeth. According to a method for illuminating teeth, an oral care illumination device is provided, for example any of the oral care illumination devices according to the inventive subject matter described above, and the oral care illumination device is positioned in the mouth of the user. When the light source of the oral care illumination device is activated, the teeth of the user are illuminated for a predetermined time, for example depending on contact time and concentration of the reactive agents used.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. An oral care illumination device, comprising:
a mouthpiece adapted to fit at least a portion of a user's mouth;
a backlight module coupled to the mouthpiece and including
at least one light source;
a light guide plate configured to receive light from the at least one light source, and including a curved light emitting surface and a plurality of microstructures;
at least one reflector to redirect light through the light guide plate; and
wherein the plurality of microstructures, the curved light emitting surface, and the at least one reflector alter a propagation of light from the at least one light source through the light guide plate to illuminate a predetermined area of the user's mouth with a desired light distribution; and
wherein the plurality of microstructures includes microstructures that are hemisphere shaped, dome shaped, pyramid shaped, sphere shaped or cube shaped.

2. The oral care illumination device of claim 1, wherein the mouthpiece includes an optically transparent teeth part allowing propagation of light from the light emitting surface of the light guide plate to the predetermined area of the user's mouth.

3. The oral care illumination device of claim 1, wherein the plurality of microstructures are configured to project a uniform distribution of light on the predetermined area of the user's mouth.

4. The oral care illumination device of claim 1, wherein the plurality of microstructures are configured to project a customized distribution of light on the predetermined area of the user's mouth.

5. The oral care illumination device of claim 1, wherein the light source is housed within the mouthpiece.

6. The oral care illumination device of claim 1, wherein the light source is mounted on a lateral side of the light guide plate.

7. The oral care illumination device of claim 1, wherein the light source is located on a front side of the backlight module.

8. The oral care illumination device of claim 1, further comprising a brightness enhancement film disposed adjacent the curved light emitting surface of the light guide plate to improve light angular uniformity.

9. A backlight module for homogenous illumination of teeth in a user's mouth, comprising:
at least one light source arranged to deliver light of a predetermined wavelength;
a light guide plate including a light emitting surface having a curvature complementary to at least a dental arch of the user's mouth, the light guide plate further including a plurality of microstructures on a light receiving surface of the light guide plate redirecting light received from the at least one light source to the light emitting surface; and
at least one reflector disposed adjacent to the light guide plate to redirect light emitted by the at least one light source through the light guide plate to the light emitting surface; and
wherein the plurality of microstructures, the curved light emitting surface and the at least one reflector alter a propagation of light from the at least one light source and illuminate the dental arch of the user's mouth with light having a desired light distribution;
wherein the plurality of microstructures includes microstructures that are hemisphere shaped, dome shaped, pyramid shaped, sphere shaped or cube shaped.

10. The backlight module of claim 9, wherein the at least one light source includes an edge-lit backlight light source.

11. The backlight module of claim 9, wherein the at least one light source includes a direct backlight light source.

12. The backlight module of claim 9, further comprising a brightness enhancement film disposed adjacent a light emitting surface of the light guide plate to improve light angular uniformity.

13. The backlight module of claim 9, wherein the plurality of microstructures are uniformly distributed over at least one light receiving surface of the light guide plate.

14. The backlight module of claim 9, wherein the plurality of microstructures are variably distributed over at least one light receiving surface of the light guide plate.

15. (canceled)
